(19) United States
(12) Patent Application Publication
(10) Pub. No.: US 2002/0164594 A1
(43) Pub. Date: Nov. 7, 2002

(54) Systematic approach to the analysis of gene function

(76) Inventors: Joseph A. Monforte, Berkeley, CA (US); James Grant Keck, Redwood City, CA (US); Frank Henri Eeckman, Berkeley, CA (US)

Correspondence Address: QUINE INTELLECTUAL PROPERTY LAW GROUP, P.C., P O BOX 458, ALAMEDA, CA, 94501, US

(21) Appl. No.: 09/811,378

(22) Filed: Mar. 16, 2001

Related U.S. Application Data
(60) Provisional application No. 60/190,406, filed on Mar. 17, 2000.
(60) Provisional application No. 60/210,927, filed on Jun. 12, 2000.

(51) Int. Cl. C12Q 1/68; G01N 33/53; C12N 5/06; C12N 15/87

(52) U.S. Cl. 435/6; 435/7.1; 435/455; 435/325

(57) ABSTRACT

In order to identify, validate, and prioritize potential therapeutic targets, it is advantageous to understand the roles that these potential therapeutic targets play within the biological systems network. Stimulation of specific sets, or matrices, of cells followed by multiple time point measurements are used to capture temporal changes exhibited by the different biochemical and genetic elements within the cells. The response of these elements to various stimuli are compared and correlated, thus identifying the functional linkages of various cellular components (for example, different genes and proteins) as different biochemical pathways are stimulated within the cell. Genetic responses are further correlated to phenotypic responses, thus providing a disease model context in which different genes play a role. The methods and cell matrices of the present invention provide a user with ways to decipher these biochemical and genetic functions, and thereby evaluate various cellular components as potential therapeutic targets. The methods and cell matrices are useful e.g., for simultaneously monitoring both the expression levels and functional state for any number of proteins in a cellular system. In addition, the methods and cell matrices of the present invention can be used, for example, to monitor the response of targeted cellular pathways to stimulation by one or more potential drug therapies. Furthermore, the methods and cell matrices are useful, for example, for evaluating potential drug candidates even when the therapeutic target has not been identified.

### CROSS-REFERENCE TO RELATED APPLICATION

This application is related to U.S. provisional patent applications Ser. Nos. 60/190,406, filed Mar. 17, 2000 and 60/210,927, filed Jun. 12, 2000. The present application claims priority to, and benefit of, U.S. Ser. No. 60/190,406 and U.S. Ser. No. 60/210,927, pursuant to 35 U. S. C. § 119(e) and any other applicable statute or rule.

### COPYRIGHT NOTIFICATION

Pursuant to 37 C.F.R. 1.71(e), Applicants note that a portion of this disclosure contains material which is subject to copyright protection. The copyright owner has no objection to the facsimile reproduction by anyone of the patent document or patent disclosure, as it appears in the Patent and Trademark Office patent file or records, but otherwise reserves all copyright rights whatsoever.

### BACKGROUND OF THE INVENTION

Functional genomics is a rapidly growing area of investigation, which includes research into genetic regulation and expression, analysis of mutations that cause changes in gene function, and development of experimental and computational methods for nucleic acid and protein analyses. The Human Genome Project has been the major catalyst driving this research; it has been through the development of high-throughput technologies that it has been possible to map and sequence complex genomes. However, while the nucleic acid sequence information elicited by these technologies represents the “structural” aspects of the genome, it is the interworkings of the genes encoded therein, and the gene products derived from these sequences, that will give a meaningful context to this information. In particular, gene expression monitoring can be utilized to examine groups of related genes, interlocking biochemical pathways, and biological networks as a whole.

### SUMMARY OF THE INVENTION

Living organisms do not exist in a static state of perfect equilibrium. Rather, they are in a constant state of metabolic flux, as they synthesize, catabolize, and generally respond to the various stimuli that constitutes their natural environment. These responses are generated within a biological systems network, which, from a pharmaceutical point of view, constitutes a vast array of potential therapeutic targets. In order to identify, validate, and prioritize these potential therapeutic targets, it is advantageous to understand the roles that these molecules play within the biological network. The present invention provides methods and biological systems, in the form of cell matrices, towards this end. Stimulation of specific sets, or matrices, of cells followed by multiple time point measurements are used to capture temporal changes exhibited by the different biochemical and genetic elements within the cells. The response of these elements to various stimuli are compared and correlated, thus identifying the functional linkages of various cellular components (for example, different genes and proteins) as different biochemical pathways are stimulated within the cell. Genetic responses are further correlated to phenotypic responses, providing a disease model context in which different genes play a role. The methods and cell matrices of the present invention provide a user with ways to decipher these biochemical and genetic functions, and thereby evaluate various cellular components as potential therapeutic targets. The methods and cell matrices are useful e.g., for simultaneously monitoring both the expression levels and functional state for any number of proteins in a cellular system. In addition, the methods and cell matrices of the present invention can be used, for example, to monitor the response of targeted cellular pathways to stimulation by one or more potential drug therapies. Furthermore, the methods and cell matrices are useful, for example, for evaluating potential drug candidates even when the therapeutic target has not been identified.

Accordingly, the present invention provides methods for deciphering genetic function. The method includes providing a plurality of cell lines, or a “matrix” of cell lines, having at least one target-specific modified cell line which differs from a corresponding parent cell line in the activity or concentration of a selected protein or nucleic acid; treating the plurality of cell lines with at least one stimulus; detecting at least one response to the stimulus; generating a plurality of profiles from data based upon the response to the stimulus; and analyzing the plurality of profiles. The plurality of cell lines can be derived from a variety of sources, including different types of tissues or tumors, primary cell lines, genetically-modified cell lines, or combinations thereof. The plurality of cell lines can contain target modified cells, or a combination of target modified cells and parent cells. The number of cell lines employed in the plurality of cell lines can vary, ranging from between about five and about fifteen parent and target-specific modified cell lines in one embodiment, to as many as 10⁴ cell lines in alternative embodiments.

The plurality of cell lines can be stimulated by a variety of compounds that affect cellular activity, including, but not limited to, DNA damaging agents; oxidative stress-inducing agents; pH-altering agents; membrane-disrupting agents; metabolic blocking agents; chemical inhibitors; chemical stimulants; ligands for cell surface receptors; antibodies; transcription promoters, enhancers, or inhibitors; translation promoters, enhancers, or inhibitors; protein-stabilizing agents; protein destabilizing agents. Changes in temperature, humidity, oxygen concentration, culture medium composition, radiation exposure, presence of additional cell types, or other environmental factors can be used to stimulate the plurality of cell lines. At least one response to the stimulus is detected, for example, by performing one or more analytical techniques such as an RNA transcription assay, protein expression assay, protein function assay, protein transportation/compartmentalization/secretion assay, phenotype-based cellular assay, metabolic assay, small molecule assay, ionic flux assay, reporter gene assay, or other assays and analytical techniques known to one skilled in the art. The assay can be performed on the cells directly, or it can be performed on some derivative of the plurality of cell lines, such as cellular lysates, extracts, or separations. Results from the detecting step are used to generate profiles for the cell lines; the resulting plurality of profiles are analyzed by any of a variety of analytical means, such as multivariate analysis, n-dimensional space analysis, principle component analysis, difference analysis, and the like. The results can be used to generate a graphical representation of the collected data across a plurality of time points.

The present invention also provides a matrix of cell lines for deciphering genetic function, having at least two target-modified cell lines, wherein the at least two target-specific modified cell lines have an altered activity or concentration of one or more selected proteins or nucleic acids as compared to one or more parent cell lines. Optionally, the matrix of cells can further comprise one or more parental cell line(s). The cell lines utilized in the matrix of the present invention can be derived from a variety of sources, including different types of tissues or tumors, primary cell lines, genetically-modified cell lines, or combinations thereof. Optionally, the matrix of cell lines is optimized for analysis of a particular disease of interest, including, but not limited to, cancer, inflammation, cardiovascular disease, diabetes, infectious diseases, proliferative diseases, immune system disorders, and central nervous system disorders.

Additionally, the present invention provides an integrated system for deciphering gene function, having (a) a plurality of cell lines differing in the activity or concentration of at least one selected protein or nucleic acid, (b) a detection system for receiving the plurality of cell lines or a derivative thereof (for example, cell lysates or chromatographic eluents), for detecting at least one response to one or more stimuli and for generating a plurality of data points, and (c) an analyzing system in operational communication with the detection system, which has a computer or computer-readable medium for organizing and analyzing the plurality of data points. Logical instructions within the computer or computer-readable medium can optionally include software for performing, for example, multivariate analysis, principle component analysis, difference analysis, or n-dimensional space analysis. The integrated system can also provide an output file.

### DETAILED DISCUSSION OF THE INVENTION

Before describing the present invention in detail, it is to be understood that this invention is not limited to particular compositions or biological systems, which can, of course, vary. It is also to be understood that the terminology used herein is for the purpose of describing particular embodiments only, and is not intended to be limiting. As used in this specification and the appended claims, the singular forms “a”, “an” and “the” include plural referents unless the content clearly dictates otherwise. Thus, for example, reference to “a device” includes a combination of two or more such devices, reference to “an analyte” includes mixtures of analytes, and the like.

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein.

The present invention describes methods of deciphering genetic function utilizing a plurality of cell lines which differ in the functional activity of a selected protein or nucleic acid. The methods includes the steps of a) providing a plurality of cell lines, or a “matrix” of cell lines, having at least one target-specific modified cell line which differs from a corresponding parent cell line in the activity or concentration of a selected protein or nucleic acid; b) treating the plurality of cell lines with at least one stimulus; c) detecting at least one response to the stimulus; d) generating a plurality of profiles from data based upon the response to the stimulus; and e) analyzing the plurality of profiles. By examining the effects generated by various stimuli, the roles that potential therapeutic targets play within the biological systems network can be elucidated, and potential therapeutic targets can be identified, validated, and/or prioritized. Optionally, the responses are measured over a period of time, reflecting the non-static nature of the biological environment.

The present invention also provides a matrix of cell lines for deciphering genetic function, having at least one parent cell line and at least two target-modified cell lines, wherein the at least two target-specific modified cell lines have an altered activity or concentration of one or more selected proteins or nucleic acids as compared to the parent cell line. Optionally, the matrix of cell lines is optimized for analysis of a particular disease of interest, including, but not limited to, cancer, inflammation, cardiovascular disease, diabetes, infectious diseases, proliferative diseases, immune system disorders, and central nervous system disorders.

Additionally, the present invention provides an integrated system for deciphering gene function, having (a) a plurality of cell lines differing in the activity or concentration of at least one selected protein or nucleic acid, (b) a detection system for receiving the plurality of cell lines or a derivative thereof (for example, cell lysates or chromatographic eluents), for detecting at least one response to one or more stimuli and for generating a plurality of data points, and (c) an analyzing system in operational communication with the detection system, which has a computer or computer-readable medium for organizing and analyzing the plurality of data points. The “operative communication” between the detection system and the analyzing system can be in the form of a person or a robotic system that conveys or transfers samples between the detection system and the analytical system. Alternatively, the equipment employed in the integrated system of the present invention can perform both the detecting and the analyzing operations.

Thus, the methods, cell matrices and integrated systems of the present invention provide a user with ways to decipher cellular biochemical and genetic functions, and thereby evaluate various cellular components as potential therapeutic targets. The methods and cell matrices are useful e.g., for simultaneously monitoring both the expression levels and functional state for any number of proteins in a cellular system. In addition, the methods and cell matrices of the present invention can be used, for example, to monitor the response of targeted cellular pathways to stimulation by one or more potential drug therapies. Furthermore, the methods and cell matrices are useful, for example, for evaluating potential drug candidates even when the therapeutic target has not been identified.

In describing and claiming the present invention, the following terminology will be used in accordance with the definitions set out below.

The term “matrix” of cell lines is used herein to describe sets of, for example, about two, four, eight, ten, fifteen, or more cell lines related in parentage and/or in a selected parameter, such as expression of a particular protein or desired phenotype.

The term “biochemical pathway” is used herein to describe any interrelated series of events or reactions; as such, this term is meant to encompass genetic pathways (series of reactions leading to induction or reduction in gene expression) as well as synthetic pathways, metabolic pathways, and the like.

### Cell-Based Matrices

The matrices of cell lines of the present invention comprise a plurality of cell lines that have been generated or selected based upon varying changes in the concentration or activity of at least one protein or nucleic acid. These plurality of cell lines are also employed in the method of the present invention, and in the integrated system described herein. The cells employed in the present invention comprise both parental cells and modified cells, including target-specific modified cells. Parental cells comprise cells which are unmodified, or “wild-type,” with respect to one or more genetic modifications. Target-specific modified cells comprise cells in which one or more modifications have been made to at least one biochemical or genetic pathway, as compared to the correlating parental cell line. These changes can result in, for example, changes in the activity or concentration of various proteins and nucleic acids, due to the integrated nature of biological systems.

The parental and modified cells include, but are not limited to, cells derived from different types of tissues or tumors, primary cell lines, cells which have been subjected to transient and/or stable genetic modification, and the like. Optionally, the cells are mammalian cells, for example murine, rodent, guinea pig, rabbit, canine, feline, primate or human cells. Alternatively, the cells can be of non-mammalian origin, derived, for example, from frogs, amphibians, or various fishes such as the zebra fish. Cells which, due to the process of “immortalization,” have been non-specifically modified can be employed as a parental cell line in the present invention. However, these immortalized cells are not considered to be “target-specific modified cells” as such, due to the imprecise nature of the changes leading to immortalization; further modification is necessary before these cells would be classified as target-specific modified cells.

Target-specific modified cells and parental cells differ by one or more modifications that have been made to at least one biochemical or genetic pathway. These modifications result in, for example, changes in the “functional activity” of at least one biological molecule, for example, a protein or a nucleic acid. A difference in the functional activity of a biological molecule refers to an alteration in an activity or a concentration of that molecule, and can include, but is not limited to, changes in transcriptional activity, translational activity, catalytic activity, binding or hybridization activity, stability, abundance, transportation, compartmentalization, secretion, or a combination thereof. The functional activity of a biological molecule can also be affected by changes in one or more chemical modifications of that molecule, including but not limited to glycosylation, phosphorylation, acetylation, methylation, ubiquitination, and the like.

The matrix of cells of the present invention comprises at least one target-specific modified cell line. In some embodiments of the present invention, between about five to about fifteen or more cell lines are employed in a given matrix of cell lines. Alternatively, as few as about two or about five cell lines, to as many as about 10³ or about 10⁴ cell lines can be used in the methods and the matrices of the present invention (optionally in a high throughput, multiwell format). The cell lines employed in the matrix can comprise various combinations of parent cells and target-specific modified cells. For example, a matrix of cell lines can have one parent cell line and a plurality of target-specific modified cell lines. Alternatively, two or three parent cell lines and a number of corresponding target-specific cell lines may be employed. Furthermore, the matrix could be composed solely of target-modified cell lines without any corresponding parent cell lines.

Cell lines which can be used in the matrix and the method of the present invention include, but are not limited to, those available from cell repositories such as the American Type Culture Collection (www.atcc.org), the World Data Center on Microorganisms (http://wdcm.nig.ac.jp), European Collection of Animal Cell Culture (www.ecacc.org) and the Japanese Cancer Research Resources Bank (http://cellbank.nihs.go.jp). These cell lines include, but are not limited to, HeLa cells, COS cells, lung carcinoma cell lines including squamous cell carcinoma cell lines (such as LK-2, LC-1, EBC-1, and NCI-H157), large cell carcinoma cell lines (such as H460 and H1299), small-cell carcinoma cell lines (such as H345, H82, H209, and N417); adenocarcinoma cell lines (such as A549, H322, H522, H358, H23 and RERF-LC-MS); fibrosarcoma cell lines (such as HT1080). Additional cell lines for use in the methods and matrices of the present invention can be obtained, for example, from cell line providers such as Clonetics Corporation (Walkersville, Md.; www.clonetics.com).

The selection of cell lines for use in the matrix depends in part upon the therapeutic target or the disease area of interest. Optionally, the collection of cells can be selected and/or optimized for the analysis of a particular biological or genetic pathway, or for cells that exhibit traits relevant to specific disease phenotypes or areas of interest. Disease areas of interest of the present invention include, but are not limited to, cancer, inflammation, cardiovascular disease, diabetes, infectious disease, proliferative diseases, immune system disorders (such as AIDS), and central nervous system disorders (for example, Alzheimer's disease and Parkinson's disease). If the target molecule is known, the modifications reflected in the matrix of cell lines can focus on this particular molecule and the pathways in which it participates. Alternatively, the plurality of cell lines can be selected for modifications made in one or more “marker” molecules that correlate to a disease-related pathway of interest.

Selective reduction or induction of the functional activity of a targeted protein (or nucleic acid) can have profound effect on other components operating either upstream or downstream within the one or more biochemical pathways that include the targeted molecule. The effects that the change in functional activity has, for example, on protein activities, protein levels, and associated transcriptional activities within the cell can be measured and used to map out both the position and the function of the various proteins within a particular pathway. Cell lines carrying specific gene knock downs or knock ins provide excellent model systems for analyzing biochemical and genetic mechanisms, particularly when the only difference among the cell lines is the alteration in the level and/or activity of a single protein or nucleic acid. These pinpoint genetic alterations provide an efficient means to decipher the roles played by various nucleic acids or proteins within the biochemical pathways in which they participate.

For example, HeLa cell lines can be finely altered to, in one circumstance, over express the p53 protein, and in another circumstance to under express c-myc. These alterations involve the insertion of exogenous elements that enable the overproduction of a protein (knockin) or reduction in the production of a constitutive protein (knockdown) within the cell. Alternatively, the targeted gene can be prevented from expressing any protein (knockout) via a number of processes including deletion of the gene or transcription promoting elements for the gene at the DNA level within the cell. An additional means for altering the functional activity of a particular protein is through mutation, wherein a targeted protein and its coding DNA sequence are modified to alter the sequence of the encoded protein in such a manner that the alteration changes the functional activity of the expressed protein.

Whether it is via knockdown, knockin, knockout or mutation, the end effect is to selectively alter the functional concentration of a targeted protein or nucleic acid. (For further information, see Berger and Kimmel, Guide to Molecular Cloning Techniques, Methods in Enzymology volume 152, Academic Press, Inc., San Diego, Calif.; and Sambrook et al., Molecular Cloning—A Laboratory Manual (2nd Ed.), Vol. 1-3, Cold Spring Harbor Laboratory, Cold Spring Harbor, N.Y., 1989). Protein and nucleic acid sequences that can be targeted in the methods of the present invention include, but are not limited to, those listed with the National Center for Biotechnology Information (www.ncbi.nlm.nih.gov) in the GenBank® databases, and sequences provided by other public or commercially-available databases (for example, the NCBI EST sequence database, the EMBL Nucleotide Sequence Database; Incyte's (Palo Alto, Calif.) LifeSeq™ database, and Celera's (Rockville, Md.) “Discovery System”™ database).

### Treatment Techniques

In the preceding step of the method of the present invention, the plurality of cell lines is generated or selected, based upon varying changes in the functional concentration of at least one protein or nucleic acid. The plurality of cell lines is then treated with at least one stimulus, in order to determine how (or whether) the cells respond in light of the difference in functional concentrations of the protein and/or nucleic acid.

A number of tools and techniques can be used in the treating step of the method of the present invention. These techniques include, but are not limited to, transient treatments with chemicals that broadly stimulate activity and/or generally perturb the environment within the cell. By “stimulation” is meant a perturbation in the equilibrium state of the biochemical and/or genetic pathways of the cell, and is not meant to be limited to an increase in concentration or biological activity. Examples of stimulatory agents, chemicals and treatments include, but are not limited to, oxidative stress, pH stress, pH altering agents, DNA damaging agents, membrane disrupters, metabolic blocking agents, and energy blockers. Additionally, cellular perturbation may be achieved by treatment with chemical inhibitors, cell surface receptor ligands, antibodies, oligonucleotides, ribozymes and/or vectors employing inducible, gene-specific knock in and knock down technologies.

The identity and use of stimulatory agents, chemicals and treatments are known to one of skill in the art. Examples of DNA damaging agents include, but are not limited to, intercalation agents such as ethidium bromide; alkylating agents such as methyl methanesulfonate; hydrogen peroxide; UV irradiation, and gamma irradiation. Examples of oxidative stress agents include, but are not limited to, hydrogen peroxide, superoxide radicals, hydroxyl free radicals, perhydroxyl radicals, peroxyl radicals, alkoxyl radicals, and the like. Examples of membrane disrupters include, but are not limited to, application of electric voltage potentials, Triton X-100, sodium dodecyl sulfate (SDS), and various detergents. Examples of metabolic blocking and/or energy blocking agents include, but are not limited to, azidothymidine (AZT), ion (e.g. Ca⁺⁺, K⁺, Na⁺) channel blockers, α and β adrenoreceptor blockers, histamine blockers, and the like. Examples of chemical inhibitors include, but are not limited to, receptor antagonists and inhibitory metabolites/catabolites (for example, mavelonate, which is a product of and in turn inhibits HMG-CoA reductase activity).

Examples of cell surface receptor ligands include, but are not limited to, various hormones (estrogen, testosterone, other steroids), growth factors, and G-protein-coupled receptor ligands. Examples of antibodies include, but are not limited to, antibodies directed against TNFα, TRAIL, or the HER2 growth factor receptor.

Examples of oligonucleotides that can be used in the treating step of the present invention include, but are not limited to, ribozymes and anti-sense oligonucleotides. Ribozymes are RNA molecules that have an enzymatic or catalytic activity against sequence-specific RNA molecules (see, for example, Intracellular Ribozyme Applications: Principles and Protocols, J. Rossi and L. Couture, eds. (1999, Horizon Scientific Press, Norfolk, UK)). Ribozymes can be generated against any number of RNA sequences, as shown in the literature for a number of target mRNAs including calretinin, TNFα, HIV-1 integrase, and the human interleukins.

Stimulatory treatments also include environment alterations such as changes in temperature, humidity, oxygen concentration, culture media composition and nutrient level, exposure to radiation, viral infection, and the introduction of other cell types to the culture. For example, a change in the nutritional content of a culture medium induces many types of cell lines to alter metabolic pathways either to compensate for the deficiency, or to decrease the energy usage of the cells.

Different stimuli or treatments potentially induce or alter a number of cellular responses which move the system away from stasis or equilibrium. Either a single stimulant or a plurality of stimulants can be used to perturb the equilibrium of the cell. Thus, in the method of the present invention, the plurality of cell lines can be exposed to, for example, more than one stimulatory agent, more than one change in an environmental parameter, or a combination of stimulatory agents and environmental alterations.

### Detection Methods

Those elements, e.g. genes, transcripts and proteins, that respond to the stimulus or move away from equilibrium, represent the interesting elements of the system with respect to deciphering genetic function and evaluating potential therapeutic targets. Either a single response or a plurality of responses can be detected and/or monitored in the method and integrated system of the present invention. In addition, the responses can be measured at either a single timepoint or over a plurality of timepoints. Optionally, at least one measurement is collected prior to stimulation.

The cellular elements that respond to a stimulus, for example, by transcriptional induction, protein activation, or changes in protein abundance, all represent potential therapeutic targets. Cellular events (responses) that are of interest and can be detected in the method of the present invention include, but are not limited to, changes in cellular transcriptional activity, cellular translational activity, activity, stability, abundance, transportation, compartmentalization, secretion, structural modification, or a combination thereof. These responses can occur and be monitored for both proteins and nucleic acids, as well as for other cellular components.

A number of different detection methods can be used to visualize and monitor these responses as they occur following stimulation of the matrix of cell lines. Such methods include, but are not limited to, RNA transcription assays, protein expression assays, protein function assays, phenotype-based cellular assays, metabolic assays, small molecule assays, ionic flux assays, reporter gene assays, membrane alteration/disruption assays, intercellular signaling assays, selective sensitivity-to-invasion assays, or a combination thereof. Many of these methodologies and analytical techniques can be found in such references as Current Protocols in Molecular Biology, F. M. Ausubel et al., eds., (a joint venture between Greene Publishing Associates, Inc. and John Wiley & Sons, Inc., supplemented through 1999), Enzyme Immunoassay, Maggio, ed. (CRC Press, Boca Raton, 1980); Laboratory Techniques in Biochemistry and Molecular Biology, T. S. Work and E. Work, eds. (Elsevier Science Publishers B. V., Amsterdam, 1985); Principles and Practice of Immunoassays, Price and Newman, eds. (Stockton Press, NY, 1991); and the like.

For example, changes in nucleic acid expression can be determined by polymerase chain reaction (PCR), ligase chain reaction (LCR), Qβ-replicase amplification, nucleic acid sequence based amplification (NASBA), and other transcription-mediated amplification techniques; differential display protocols; analysis of northern blots, enzyme linked assays, micro-arrays and the like. Examples of these techniques can be found in, for example, PCR Protocols A Guide to Methods and Applications (Innis et al. eds) Academic Press Inc. San Diego, Calif. (1990).

Alternatively, the expression pattern of genes can be rapidly analyzed as described by Wang et al. (Nucleic Acids Research (1999) vol. 27, pages 4609-4618). This technique employs PCR amplification of cDNAs which have been cleaved by frequently-cutting endonucleases, such as DpnII and NlaIII, and primed with defined sequences prior to amplification.

Another method for detecting molecular events within the plurality of cell lines utilizes real-time PCR, using, for example, molecular beacons or FRET (fluorescence resonance energy transfer). The FRET technique utilizes molecules having a combination of fluorescent labels which, when in proximity to one another, allows for the transfer of energy between labels (see, for example, X. Chen and P. -Y. Kwok, (1997) Nucleic Acid Research vol. 25, pp. 2347-2353).

Optionally, the responses of the plurality of cell lines can be monitored by fluorescence activated cell sorting, or FACS. A wide variety of flow-cytometry methods have been published. For a general overview of fluorescence activated flow cytometry see, for example, Abbas et al. (1991) Cellular and Molecular Immunology, W. B. Saunders Company; Coligan et al. (eds)(1991) Current Protocols in Immunology, and Supplements, John Wiley and Sons, Inc. (New York); and Kuby (1992) Immunology, W. H. Freeman and Company,. Fluorescence activated cell scanning and sorting devices are available from several companies, including, e.g., Becton Dickinson and Coulter.

Alternatively, high throughput screening systems utilizing microfluidic technologies, available, for example, from Agilent/Hewlett Packard (Palo Alto, Calif.) and Caliper Technologies Corp. (Mountain View, Calif.) could be employed for detecting the response(s) generated in the plurality of cell lines. The Caliper Lab Chip™ technology uses microscale microfluidic techniques for performing analytical operations such as the separation, sizing, quantification and identification of nucleic acids (for further information, see www.calipertech.com).

### Generation of Profiles

Observation of cellular events as they occur over time and in response to one or more stimuli provides a dynamic view of the biomolecular activity of the cell. These cellular events, or responses, are evaluated and recorded for comparison. This is achieved by collecting the plurality of data points representing information related to the plurality of cell lines and the one or more responses of the cellular system to the at least one stimulus.

For each experiment performed, the plurality of data points is gathered into a database and used to generate a “profile” for the corresponding cell line. The plurality of data points representing the cellular responses to stimulation can be linear or nonlinear. In one embodiment of the present invention, the generating the plurality of profiles consists of a) selecting a first cell line from the plurality of cell lines; b) evaluating at least one response, and optionally multiple responses; c) recording the evaluation of the at least one response; and d) repeating these steps for additional cell lines in the plurality of cell lines. In another embodiment of the method of the present invention, the evaluating and recording of information is performed on the entire plurality of cell lines simultaneously. During the recording step, the response (or responses) generated for each cell line are entered into a profile database for further analysis. The entire set of cell lines can be evaluated for response to a stimulus, or a subset of the set of cell lines can be examined.

Generation of the plurality of profiles for the plurality of cell lines generally results in a large quantity of data reflecting information related to the cell types used and the responses measured for the plurality of cell lines. In one embodiment of the method of the present invention, the plurality of data points is entered as character strings, or as descriptors, into a database. The character strings or descriptors can be used to encode include any relevant information derived from or detected within the plurality of cell lines, including any physical characteristics, activities, or other information related to the cell types used and the responses detected. In general, the database is embodied in a computer or computer readable medium and can be accessed by a user and/or integrated system.

### Data Analysis

The information encoded in the database (i.e. the plurality of profiles) can then be evaluated in the analyzing step of the method of the present invention. Analysis of the data involves the use of a number of statistical tools to evaluate the measured responses and changes based on type of change, direction of change, shape of the curve in the change, timing of the change and amplitude of change. This information can be used to perceive and interpret the impact that alterations, ranging from a “minor” change in a single nucleotide to major permutations in one or more metabolic pathway, can have on the biological systems network as a whole.

Multivariate statistics, such as principal components analysis (PCA), factor analysis, cluster analysis, n-dimensional analysis, difference analysis, multidimensional scaling, discriminant analysis, and correspondence analysis, can be employed to simultaneously examine multiple variables for one or more patterns of relationships (for a general review, see Chatfield and Collins, “Introduction to Multivariate Analysis,” published 1980 by Chapman and Hall, New York; and Höskuldsson Agnar, “Predictions Methods in Science and Technology,” published 1996 by John Wiley and Sons, New York). Multivariate data analyses are used for a variety of applications involving these multiple factors, including quality control, process optimization, and formulation determinations. The analyses can be used to determine whether there are any trends in the data collected, whether the properties or responses measured are related to one another, and which properties are most relevant in a given context (for example, a disease state). Software for statistical analysis is commonly available, e.g., from Partek Inc. (St. Peters, Mo.; see www.partek.com).

Multivariate statistics is particularly useful for determination and analysis of polygenic effects within a cell line. One common method of multivariate analysis is principal component analysis (PCA, also known as a Karhunen-Loeve expansion or Eigen-XY analysis). PCA can be used to transform a large number of (possibly) correlated variables into a smaller number of uncorrelated variables, termed “principal components.” Multivariate analyses such as PCA are known to one of skill in the art, and can be found, for example, in Roweis and Saul (2000) Science 290:2323-2326 and Tenenbaum et al. (2000) Science 290:2319-2322.

The responses generated by a given plurality of cell lines can be grouped, or clustered, using multivariate statistics. Clusters for each different stimulation (treating) and observation (detecting) experiment are compared and a secondary set of correlations/noncorrelations are made. Based on these different sets of correlations, a network map can be created wherein the relative relationships of the different genetic elements can be established as well as how they may act in concert. In addition, the data can be visualized using graphical representations. Thus, the temporal changes exhibited by the different biochemical and genetic elements within a genetically-related group of cells lines can be transformed into information reflecting the functioning of the cells within a given environment.

### Integrated System Components

The present invention also provides an integrated system for deciphering gene function. The integrated system includes a plurality of cell lines differing in the activity or concentration of at least one selected protein or nucleic acid. As previously described for the matrix of cells of the present invention, the plurality of cell lines employed in the integrated system comprise at least one target-specific modified cell line, and can include, but are not limited to, cells derived from different types of tissues or tumors, primary cell lines, cells which have been subjected to transient and/or stable genetic modification, and the like.

In addition, the integrated system has a detection system, which performs several functions. First, the detection system receives the plurality of cell lines. The detection system can accommodate whole cells, or a derivative thereof, for example, cell lysates or chromatographic eluents. Optionally, the detection system receives the plurality of cell lines in a multi-well container, such as a 96, 384, 768 or 1536 well plates (available from various suppliers such as VWR Scientific Products, West Chester, Pa.). The multi-well container can be a receptacle in which the treating or stimulating event takes place. Additionally the multi-well container can accommodate further manipulations to the plurality of cell lines, such as generation of the cell line derivatives.

The detection system detects at least one response to one or more stimuli. The cell lines can be stimulated prior to insertion into the detection system, or after insertion. Detection of the at least one response can be achieved by a number of analytical techniques such as mass spectrometry; NMR spectroscopy; visible/UV/infra-red spectroscopy; fluorescence, phosphorescence, chemiluminescence and/or other types of photoemission spectroscopy (using either static or time-resolved methodologies); potentiometry, calorimetry; radiography; diffraction methodologies; and electron-pair resonance (EPR) spectroscopy, optionally coupled with techniques such as chromatography, electrophoresis (including capillary electrophoresis), microscopy, cytometry, and the like.

Additionally, the detection system generates a plurality of data points based upon both information related to the plurality of cell lines and the at least one response to the one or more stimuli. The data generated can include, but are not limited to, information related to cell type(s), gene sequences, genetic polymorphism, mRNA expression levels, mRNA splicing and/or modification events (such as polyadenylation, removal of leader sequences, and capping), transcript transportation events, mRNA expression ratios, protein expression levels, protein activity levels, protein modification levels, protein-protein interactions, reporter gene expressions/activities, protein transportation, localization and secretion events (including cross membrane and extracellular transport), cellular phenotypic alterations (including alterations in cell morphology), cellular properties (such as adhesion, nonadhesion, differentiation, invasion, proliferation, cell-cell interaction, synchronization, and termination), changes in cellular factors (including ionic and energy levels), and other observable changes that occur within cells.

Furthermore, the integrated system of the present invention has a data analyzing system in operational communication with the detection system. The data analyzing system comprises a computer or computer-readable medium having one or more logical instructions for organizing the plurality of data points into a database and one or more logical instructions for analyzing the plurality of data points. Optionally, the data analyzing system can also have one or more logical instructions for operating components of the detection system, and can be accessed by a user and/or the integrated system. The data analyzing system can be a computer running any available operating system (commercial or otherwise), or it can be another form of computational device known to one of skill in the art. Software for manipulating information descriptor elements is available, or can easily be constructed by one of skill using a standard programming language such as C, C++, Visual Basic, Fortran, Basic, Java, or the like. For example, a computer system can include software having descriptors of the data points, optionally modified for conjunction with a user interface (e.g., a GUI in a standard operating system such as a Windows, Macintosh, UNIX, LINUX, and the like), to manipulate the strings of characters or descriptors representing the plurality of profiles. Standard desktop applications including, but not limited to, word processing software (e.g., Microsoft Word™ or Corel WordPerfect™), spreadsheet and/or database software (e.g., Microsoft Excel™, Corel Quattro PrO™, Microsoft Access™, Paradox™, Filemaker PrO™, Oracle™, Sybase™, and Informix™) can be adapted for generating, storing and/or analyzing the plurality of profiles.

The character strings or descriptors can be used to encode any relevant information derived from or detected within the plurality of cell lines, including any physical characteristics, activities, or other information related to the cell types used and the responses detected. The logical instructions within the computer or computer-readable medium can optionally include software for performing, for example, multivariate analysis, principle component analysis, difference analysis, or n-dimensional space analysis. In addition, the integrated system can also provide an output file. The output file can be in the form of a graphical representation of part or all of the plurality of data points. Alternatively, the output file can comprise descriptors, for example, for entering this information into an alternative database or computer-readable medium.

### Kits

In an additional aspect, the present invention provides kits embodying the methods and devices herein. Kits of the invention optionally comprise one or more of the following elements: (1)one or more target-specific modified cell lines (optionally two or more target-specific cell lines); (2) one or more parent cell lines; (3) one or more assay components, including, but not limited to buffers, substrates, cofactors, inhibitors, and the like; (4) a computer or computer-readable medium for storing and/or evaluating the assay results; (5) logical instructions for practicing the methods described herein; (6) logical instructions for analyzing and/or evaluating the assay results as generated by the methods herein, and, optionally, (7) packaging materials.

### Uses of the Methods, Devices and Compositions of the Present Invention

Modifications can be made to the method and materials as described above without departing from the spirit or scope of the invention as claimed, and the invention can be put to a number of different uses, including:

The use of any method herein, to analyze genetic function.

The use of any integrated system, or any cell matrix as described herein, to analyze genetic function.

An assay, kit or system utilizing a use of any one of the selection strategies, materials, components, cell matrices, methods or substrates hereinbefore described. Kits will optionally additionally include instructions for performing the methods or assays, packaging materials, one or more containers which contain assay, device or system components, or the like.

In a further aspect, the present invention provides for the use of any component or kit herein, for the practice of any method or assay herein, and/or for the use of any apparatus or kit to practice any assay or method herein.

While the foregoing invention has been described in some detail for purposes of clarity and understanding, it will be clear to one skilled in the art from a reading of this disclosure that various changes in form and detail can be made without departing from the true scope of the present invention. For example, all the methods and compositions described above may be used in various combinations. All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods, and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims. All publications, patents, patent applications, Internet citations, and/or other documents cited in this application are incorporated by reference in their entirety for all purposes to the same extent as if each individual publication, patent, patent application, Internet citation and/or other document were individually indicated to be incorporated by reference for all purposes.

What is claimed is:

1. A method of deciphering genetic function, the method comprising:
providing a plurality of cell lines comprising at least one target-specific modified cell line, wherein the at least one target-specific modified cell line and a corresponding parent cell line differ in the activity or concentration of a selected protein or nucleic acid;
treating the plurality of cell lines with at least one stimulus;
detecting at least one response to the at least one stimulus for the plurality of cell lines;
generating a plurality of profiles for the plurality of cell lines, which plurality of profiles comprises data based upon the at least one response to the stimulus; and
analyzing the plurality of profiles.

2. The method of claim 1, wherein the step of providing a plurality of cell lines comprises providing parent cell lines derived from different types of tissues or tumors, primary cell lines, genetically-modified cell lines, or combinations thereof.

3. The method of claim 1, wherein the plurality of cell lines comprises target-specific modified cell lines.

4. The method of claim 1, wherein the plurality of cell lines comprises between about two and about 100,000 cell lines.

5. The method of claim 4, wherein the plurality of cell lines comprises between about five and about 10,000 cell lines.

6. The method of claim 5, wherein the plurality of cells lines comprises about ten to about 500 cell lines.

7. The method of claim 1, wherein the plurality of cell lines comprises about five to about fifteen cell lines.

8. The method of claim 1, wherein the plurality of cell lines comprises target-specific modified cell lines and parent cell lines.

9. The method of claim 8, wherein each parent cell line corresponds to at least two target-specific modified cell lines.

10. The method of claim 9, wherein each parent cell line corresponds to at least five target-specific modified cell lines.

11. The method of claim 8, wherein the plurality of cell lines comprises a single parent cell line and multiple target-specific modified cell lines.

12. The method of claim 11, wherein the plurality of cell lines comprises a single parent cell line and two target-specific modified cell lines.

13. The method of claim 11, wherein the plurality of cell lines comprises a single parent cell line and between about two to about 100,000 target-specific modified cell lines.

14. The method of claim 11, wherein the plurality of cell lines comprises a single parent cell line and between about five to about fifteen target-specific modified cell lines.

15. The method of claim 1, wherein the step of treating comprises stimulating the plurality of cell lines with a compound that affects a cellular activity.

16. The method of claim 15, wherein the compound that affects the cellular activity comprises DNA damaging agents; oxidative stress-inducing agents; pH-altering agents; membrane-disrupting agents; metabolic blocking agents; chemical inhibitors; ligands for cell surface receptors; antibodies; transcription promoters, enhancers, or inhibitors; translation promoters, enhancers, or inhibitors; protein-stabilizing agents; protein destabilizing agents; or combinations thereof.

17. The method of claim 1, the step of treating comprising stimulating the plurality of cell lines by altering an environmental parameter.

18. The method of claim 17, wherein the environmental parameter comprises temperature, humidity, oxygen concentration, culture medium composition, exposure to radiation, exposure to additional cell types, or a combination thereof.

19. The method of claim 1, wherein the step of treating comprises perturbing the plurality of cell lines with a plurality of stimuli, and wherein the plurality of stimuli comprises one or more compounds that affects cellular activity, an alteration in one or more environmental parameters, or combinations thereof.

20. The method of claim 1, wherein the step of detecting at least one response comprises performing an analytical technique comprising an RNA transcription assay, a protein expression assay, a protein function assay, a phenotype-based cellular assay, a metabolic assay, a small molecule assay, an ionic flux assay, a reporter gene assay, or a combination thereof.

21. The method of claim 20, wherein the step of detecting at least one response further comprises detecting a change in cellular transcriptional activity, cellular translational activity, nucleic acid splicing or modification activity, nucleic acid binding activity, protein activity, protein stability, protein abundance, protein transportation, protein compartmentalization, protein secretion, protein modification, or a combination thereof.

22. The method of claim 1, wherein the step of detecting at least one response comprises performing a fluorescence-assisted cell sorting (FACS) assay

23. The method of claim 1, wherein the step of detecting at least one response comprises detecting a polygenic effect.

24. The method of claim 1, further comprising detecting at least one cellular activity in the plurality of cell lines prior to treating the plurality of cell lines.

25. The method of claim 1, wherein the step of generating a plurality of profiles for the plurality of cell lines comprises generating a profile for each member of the plurality of cell lines.

26. The method of claim 1, wherein the step of generating the plurality of profiles comprises
selecting a first cell line from the plurality of cell lines;
evaluating the at least one response in the first cell line;
recording the evaluation of the at least one response in a profile database; and
repeating the selecting, evaluating and recording for multiple cell lines in the plurality of cell lines.

27. The method of claim 26, wherein the step of evaluating the at least one response comprises measuring data at a plurality of time points.

28. The method of claim 27, wherein the step of analyzing the plurality of profiles comprises generating a graphical representation of the data and the plurality of time points.

29. The method of claim 27, wherein the step of analyzing the plurality of profiles comprises performing multivariate analysis for the data.

30. The method of claim 27, wherein the step of analyzing the plurality of profiles comprises analyzing the data in n-dimensional space.

31. The method of claim 27, wherein the step of analyzing the plurality of profiles comprises performing principle component analysis.

32. The method of claim 27, wherein the step of analyzing the plurality of profiles comprises performing a difference analysis.

33. The method of claim 27, the method further comprising:
building a network model from the data.

34. A matrix of cell lines for deciphering genetic function, the matrix comprising at least two target-specific modified cell lines, wherein the at least two target-specific modified cell lines have an altered activity or concentration of a selected protein or nucleic acid as compared to a parent cell line.

35. The matrix of claim 34, wherein the matrix of cell lines further comprises a parent cell line.

36. The matrix of claim 34, wherein the matrix of cell lines comprises cell lines optimized for the analysis of a particular disease area of interest.

37. The matrix of claim 36, wherein the particular disease area of interest comprises cancer, inflammation, cardiovascular disease, diabetes, infectious diseases, proliferative diseases, an immune system disorder, or a central nervous system disorder.

38. The matrix of claim 34, wherein the cell lines are selected from the group consisting of cell lines derived from different types of tissues or tumors, primary cell lines, cell lines comprising stable or transient genetic modifications, and combinations thereof.

39. The matrix of claim 34, wherein the cell lines comprise mammalian cells.

40. The matrix of claim 34, wherein the altered activity or concentration of the selected protein or nucleic acid comprises a change in a cellular transcriptional activity, a cellular translational activity, a nucleic acid splicing or modification activity, a protein activity, a protein stability, a protein abundance, a transportation activity, a protein compartmentalization, protein secretion, or a combination thereof.

41. The matrix of claim 34, wherein the parent cell line has between about five and about fifteen corresponding target-specific modified cell lines.

42. A matrix of cell lines for deciphering genetic function, the matrix comprising:
at least two parent cell lines; and
at least two target-specific modified cell lines;
wherein each parent cell line has at least one corresponding target-specific modified cell line, and wherein the corresponding target-specific modified cell line has an altered activity or concentration of a selected protein or nucleic acid as compared to the parent cell line.

43. The matrix of claim 42, wherein the matrix of cell lines comprises cell lines optimized for the analysis of a particular disease area of interest.

44. The matrix of claim 43, wherein the particular disease area of interest comprises cancer, inflammation, cardiovascular disease, diabetes, infectious diseases, proliferative diseases, an immune system disorder, or a neurological disorder.

45. The matrix of claim 42, wherein the cell lines are selected from the group consisting of cell lines derived from different types of tissues or tumors, primary cell lines, cell lines comprising stable or transient genetic modifications, and combinations thereof.

46. The matrix of claim 42, wherein the cell lines comprise mammalian cells.

47. The matrix of claim 42, wherein the altered activity or concentration of the selected protein or nucleic acid comprises a change in a cellular transcriptional activity, a cellular translational activity, a protein activity, a protein stability, a protein abundance, protein compartmentalization, a protein modification, or a combination thereof.

48. The matrix of claim 42, wherein each parent cell line has at least two corresponding target-specific modified cell lines.

49. The matrix of claim 48, wherein each parent cell line has at least five corresponding target-specific modified cell lines.

50. The matrix of claim 42, wherein the matrix of cell lines further comprises at least three parent cell lines and at least three target-specific modified cell lines, wherein each parent cell line has at least one corresponding target-specific modified cell line.

51. An integrated system for deciphering gene function comprising:
a plurality of cell lines comprising at least one target-specific modified cell line, wherein the at least one target-specific modified cell line and a corresponding parent cell line differ in the activity or concentration of a selected protein or nucleic acid;
a detection system for receiving the plurality of cell lines or a derivative thereof, wherein the detection system detects at least one response to one or more stimuli for at least two of the plurality of cell lines or derivative thereof, and generates a plurality of data points based upon the at least one response to the one or more stimuli; and
a data analyzing system in operational communication with the detection system, the data analyzing system comprising a computer or computer-readable medium comprising one or more logical instructions for organizing the plurality of data points into a database and one or more logical instructions for analyzing the plurality of data points.

52. The integrated system of claim 5 1, wherein the detection system detects a signal or a result from an analytical technique.

53. The integrated system of claim 52, wherein the analytical technique comprises an RNA transcription assay, a protein expression assay, a protein function assay, a phenotype-based cellular assay, a metabolic assay, a cofactor or small molecule assay, an ionic potential-measuring assay, a reporter gene assay, or a combination thereof.

54. The integrated system of claim 51, wherein the detection system detects the at least one response at a plurality of time points.

55. The integrated system of claim 5 1, wherein the detection system detects a plurality of responses at a plurality of time points.

56. The integrated system of claim 5 1, wherein the database comprises a plurality of profiles for the plurality of cell lines.

57. The integrated system of claim 51, wherein the one or more logical instructions for analyzing the plurality of data points comprises software for generating a graphical representation of the plurality of responses and the plurality of time points.

58. The integrated system of claim 5 1, wherein the one or more logical instructions for analyzing the plurality of data points comprises software for performing multivariate analysis for the plurality of data points.

59. The integrated system of claim 5 1, wherein the one or more logical instructions for analyzing the plurality of data points comprises software for analyzing the plurality of data points in n-dimensional space.

60. The integrated system of claim 51, wherein the one or more logical instructions for analyzing the plurality of data points comprises software for performing principle component analysis upon the plurality of data points.

61. The integrated system of claim 51, wherein the one or more logical instructions for analyzing the plurality of data points comprises software for performing difference analysis upon the plurality of data points.

62. The integrated system of claim 5 1, further comprising an output file.

63. The integrated system of claim 62, wherein the output file comprises a network model of the plurality of data.

64. A kit for deciphering genetic function, the kit comprising:
two or more target-specific modified cell lines;
one or more buffers; and
one or more substrates.

65. The kit of claim 64, further comprising one or more parent cell lines.

66. The kit of claim 64, wherein the two or more target-modified cell lines comprises between about ten and about fifteen target-modified cell lines.

67. The kit of claim 64, further comprising software for storing and analyzing data.
